(19) United States
(12) Patent Application Publication
(10) Pub. No.: US 2008/0255579 A1
(43) Pub. Date: Oct. 16, 2008

(54) Implantation Tool for Intraocular Lenses

(76) Inventors: Peter Wollenhaupt, Selm (DE); Gerhard Merten, Lunen (DE); Jochen Kammann, Davos-Platz (CH)

Correspondence Address: BAKER & MCKENZIE LLP, 1114 AVENUE OF THE AMERICAS, NEW YORK, NY, 10036, US

(21) Appl. No.: 12/066,250

(22) PCT Filed: Sep. 6, 2006
(86) PCT No.: PCT/DE2006/001557

§ 371 Date: Mar. 7, 2008

(30) Foreign Application Priority Data
Sep. 9, 2005 (DE) 10 2005 042 849.5

(51) Int. Cl. A61F 9/007

(52) U.S. Cl. 606/107

(57) ABSTRACT

The object of the invention is to permit a smooth advance movement of intraocular lenses (25) by means of a compact design of the implantation tool (1) and a short operating cycle, the push rod in the main body (2) of the implantation tool (1) being driven by a two-stage hydraulic mechanism (3), wherein a connectable cannula (23) with roller (24) forms the sterile storage container for the intraocular lens (25) and, after replacement of the sterile storage fluid by a lubricant fluid, gently transports it into the eye ready for implantation.

The invention relates to a surgical tool for implanting an intraocular lens, in short IOL, into the human eve.

### BACKGROUND OF THE INVENTION

IOLs are used to replace the natural lens of the eye or to correct the refractive power while retaining the natural lens and consist of the artificial optic and the socalled haptic. The haptic forms the carrying element for positioning the IOLs in the capsular bag sulcus ciliaris and the anterior chamber of the eye. The IOLs preferably consist of soft foldable silicone, acrylic or hydrogel und can be of different designs. Due to these materials it is possible to fold or roll up such soft IOLs and to insert them into the eye through a small incision in the eye which shall not be larger than 2-3 mm. Said small incision serves for cataract surgery using phacoemulsification and aspiration where the natural lens is broken up by a special ultrasound-activated needle and the fragment is removed through a small cannula.

A number of different methods and devices for folding and rolling up and advancing the IOLs have already been employed all of which are aimed at avoiding damage to the optic and/or haptic. Reference is made to a representative prior art as it is known by EP 962195 B1, EP 966238 B1, and DE 40 30492 C1 disclosing implantation tools with cannulas connected upstream which are equipped with IOLs adapted to the respective visual impairment. The implantation tools are of cylindrical shape and are equipped with axially loaded pistons with piston rod and upstream soft tappet. By means of the tappets the IOLs are transported from the cannula into the eye. As a rule, the the piston rods are operated by the surgeon applying finger pressure, but manual and motor-driven rotary drives have also been in use. Particularly the motor-driven version of the rotary drive had been aimed at achieving a constant advancing speed, an accurate insertion and gentle handling of the IOL.

When handling the known implantation tools it has proved disadvantageous that the relatively long dimensioning of these tools brings with it unfavourable lever conditions for the surgeon leading to relatively large undesired movements in the area of the eye where the incision is made thus complicating the operation. By U.S. Pat. No. 4,699,140 an implantation tool is known which achieves the axial movement of the piston rod by a radial movement of a lever including a cam surface. A smooth transport movement of the IOL to be implanted can obviously not be ensured here.

By U.S. Pat. No. 4,976,716 furthermore an implantation tool is known which is also aimed at advancing the piston rod by radially moving a lever wherein the radial movement is to be converted into an axial movement via a ratchet mechanism. A smooth movement can by no means be achieved with this arrangement.

### OBJECT OF THE INVENTION

The object of the invention is to create an implantation tool which permits a smooth advance movement of the IOL, a compact design and a reduction of the operation cycle.

The object according to the invention is achieved by means of the characteristic features of claim 1. The preferred design types and advantageous solutions result from the subclaims and the description. According to the invention a surgical implantation tool is created for sterily implanting a foldable IOL made of soft pliable material, particularly silicone, acrylic or hydrogel, into an eye. This tool is provided with a tubular main body, with a tubular implant tool which has its internal diameter matched to the Tens and which allows to insert the lens through an incision in the eye into the lens capsule of the eye, with a tappet and a pushing device for pushing the lens out of the implant tool by means of the tappet. According to the invention the pushing device is equipped with a two-stage hydraulic mechanism as drive for the tappet or driven by a two-stage hydraulic mechanism.

### ADVANTAGES OF THE INVENTION

The advantage of the solution according to the invention is seen in that the IOL can be transported into the lens capsule of the eye or any other desired locations smoothly, in a controlled manner and accurately with minimal effort and thanks to a roller path in the area of the cannula a particularly gentle advance movement with the IOL in the smallest rolled-up dimension possible can be achieved. The decisive advantage will come from the implantation process being more precise and accelerated. In addition, the IOL can be already pre-loaded or added in sterile condition.

### EXPLANATION OF THE INVENTION

The object of the invention is to permit a smooth advance movement of intraocular lenses by means of a compact design of the implantation tool and a short operating cycle, the push rod in the main body of the implantation tool being driven by a two-stage hydraulic mechanism, wherein preferably a connectable cannula with roller path forms the sterile storage container for the intraocular lens and gently transports it into the eye ready for implantation consequently forthwith forming the implant tool.

The implantation tool is divided primarily into a main body and a cannula. The cannula is connected with the main body by a simple, preferably detachable, connection such as, for example, a bayonet catch or a T-guide with snap. The cannula forms a storage container and at the same time a transport rollerway for the IOL. The IOL with its haptic is stored in a sterile fluid in the cannula. The cannula is closed at its front end with a detachable cap. The sterile fluid will be replaced by a lubricant fluid to be added for the IOL. This lubricant fluid at the same time serves for stabilizing the different compartments. The back end of the cannula is closed by a film. In the center of the canula the tappet being provided with a flexible front end goes through the film bringing it in advancing contact with the IOL. By axially operating the piston-end tappet section which projects from the main body by a defined stop dimension the IOL is transported through the roller path to short of the point where it will leave the cannula. The fluid displaced by the piston during this movement is pressed via a pilot check valve both into the rearward piston annulus and the radial piston annulus of the second hydraulic stage.

As advancing the IOL by means of the tappets with their flexible ends used in the past repeatedly led to damage to the lens a solution is sought according to a further feature of the invention ensuring a problem-free advancement of the lens. For this purpose, a piston cylinder unit equipped with two filling hoses and containing a lubricant fluid is assigned to the implantation tool. By alternatingly operating the piston rod equipped with two pistons and connecting the filling hoses at the cannula tip on the one hand and at the cannula end on the other hand a replacement of the sterile storage fluid by the lubricant is obtained shortly before the operation. This exchange of fluid is coordinated such that firstly the storage fluid is pressed out of the cannula end by the lubricant and subsequently, the lubricant introduced is removed again in the direction of the cannula tip and at the same time lubricant pressed in at the cannula end. This process is synchronous with the tappet of the implantation tool retracting. During this pushing process a hydrostatic underpressure occurs in front of the IOL and a hydrostatic overpressure behind the IOL which causes the IOL to be transported into its rolled exit position without any significant pressure load being exerted on the IOL by the tappet of the implantation tool. Only in the completely rolled-up most resistant condition will the IOL be transported by the tappet through the cannula into the eye. This only requires the surgeon to radially actuate the second hydraulic stage.

The surgeon, after removing the natural lens through the 3 mm maximum incision made before, thus only needs a piston stroke of about 10 mm for inserting the cannula and operating the radial piston. The IOL rolled up to its smallest size leaves the cannula at the tip, unrolls and is positioned by means of the haptic in the lens capsule of the eye or any other locations, if necessary.

In the following, the invention is explained by way of a typical embodiment on the basis of sectional drawings.

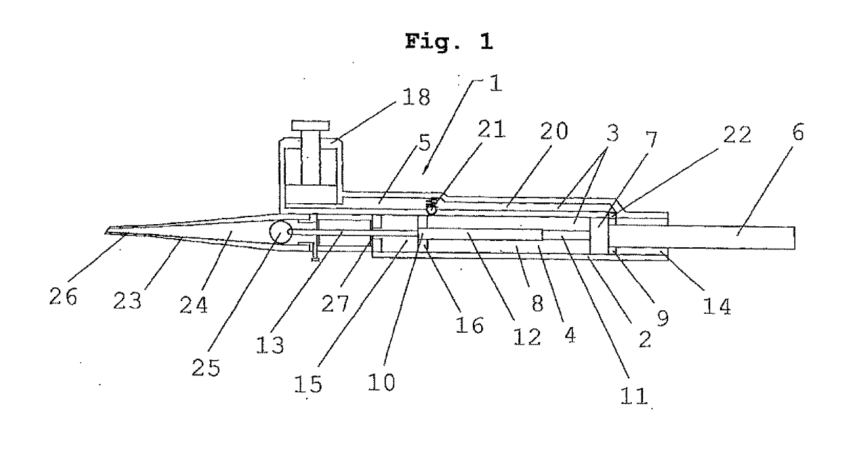

FIG. 1 shows the implantation tool with the flanged-on cannula and the IOL embedded therein in the initial position.

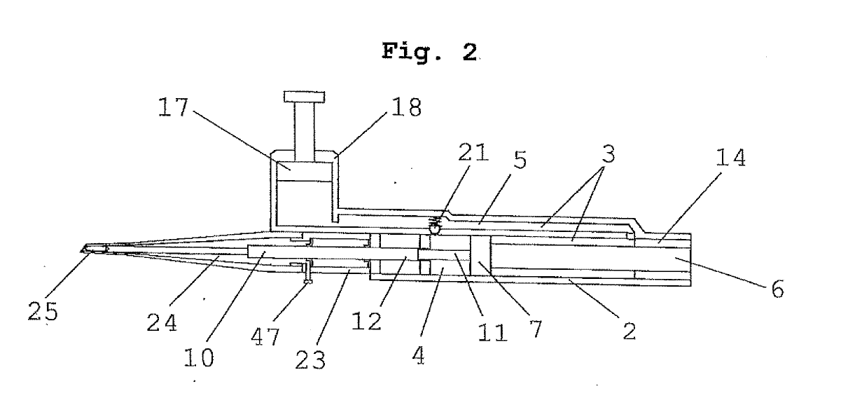

FIG. 2 shows the implantation tool after the manual axial operation up to the rear stop and the position of the IOL before it leaves the cannula.

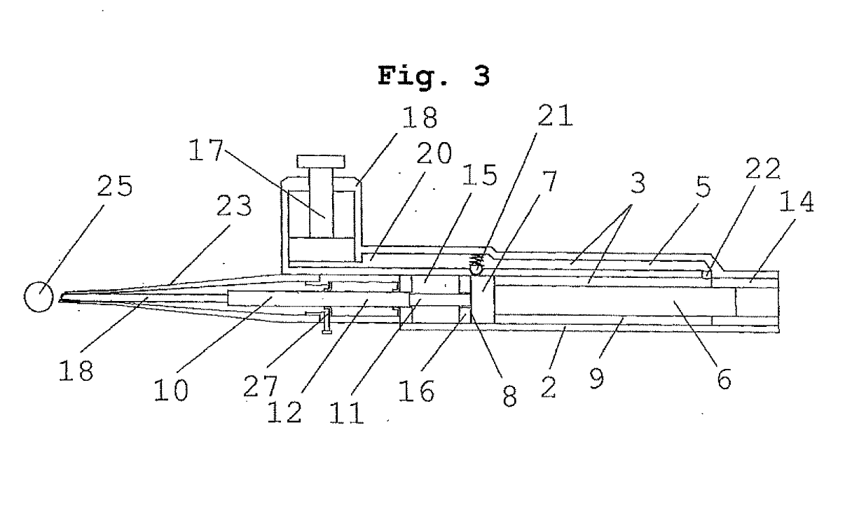

FIG. 3 shows the implantation tool in the end position after operation of the radially acting piston and the IOL having left the cannula.

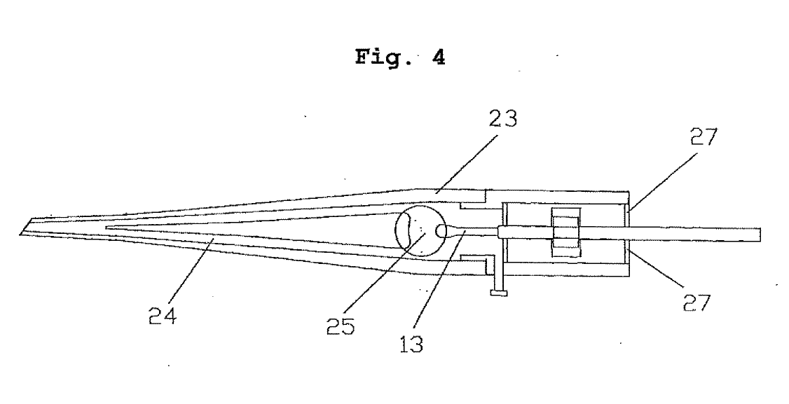

FIG. 4 shows a simplified representation of the cannula in its initial position with the tappet in contact with the IOL.

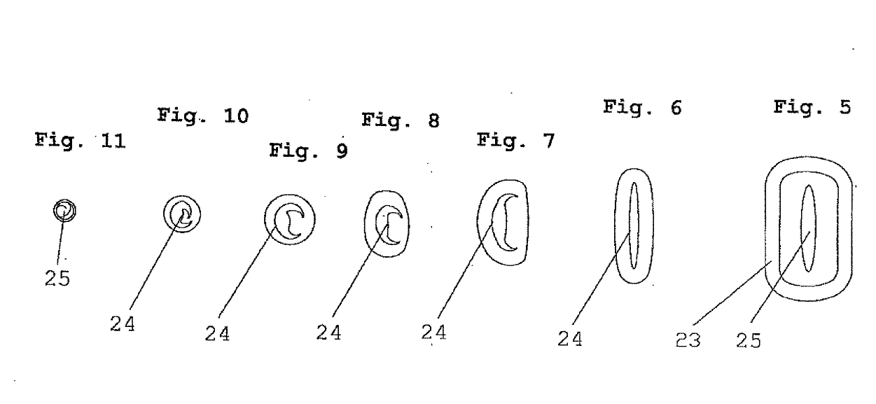

FIGS. 5-11 show the different phases of passage of the IOL with its haptic through the roller path of the cannula from the start of transport to the start of exit.

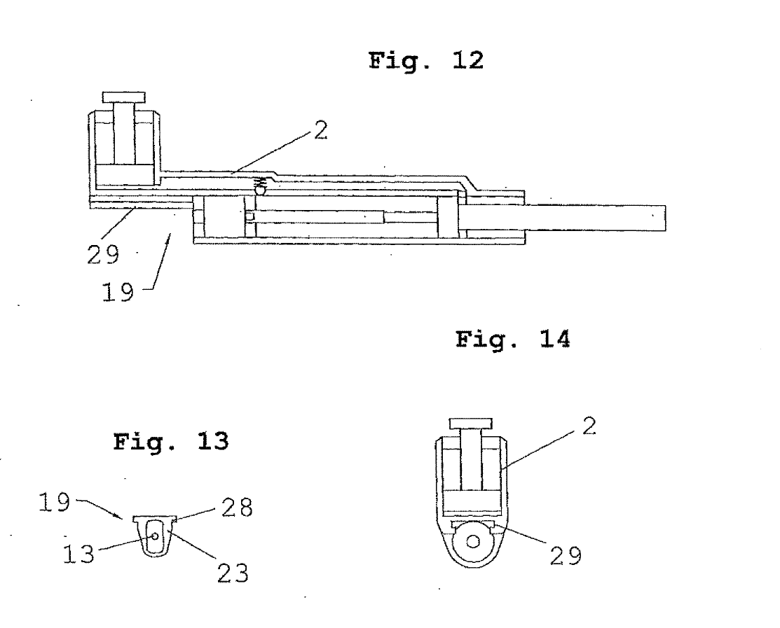

FIG. 12 shows the implantation tool with a T-guide adapted to accommodate the cannula.

FIG. 13 shows the cross-section of the cannula in the area of the T-guide with lateral sliding rails.

FIG. 14 shows the cross-section of the implantation tool in the area of the T-guide adapted to accommodate the cannula.

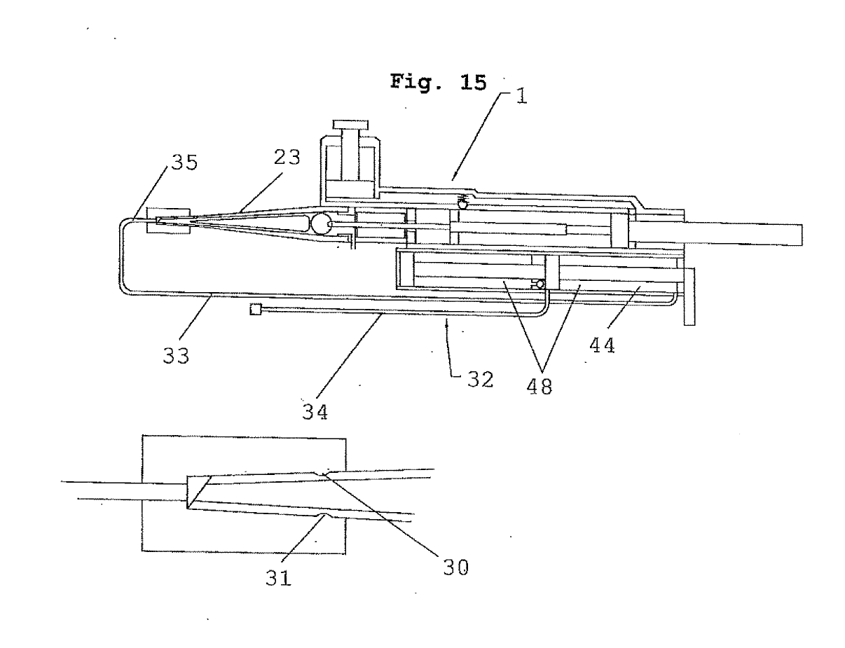

FIGS. 15-18 show the implantation tool with a lubricant fluid container in different stages of use.

FIG. 1 depicts an implantation tool I with flanged-on cannula 23 and the IOL 25 embedded therein. The cannula 23 preferably directly forms the implant tool. The implant tool can, however, also be fixable to the implantation tool I by means of an adapter or the like. The implantation tool 1 comprises a main body 2 including a two-stage hydraulic mechanism 3 which is composed of the first hydraulic stage 4 and the second hydraulic stage 5. Guided in the tubular main body 2 is a piston rod 6 with piston 7 and a three-stage tappet 10, the latter being comprised of a piston-end tappet section 11, a center tappet section 12, and a front tappet section 13. The piston-end tappet section 11 of the tappet 10 has a clearly smaller diameter than the piston rod 6. The center tappet section 12 of the tappet 10, on the other hand, has a larger diameter than tappet section 11. The front tappet section 13 of the tappet 10, however, has the smallest diameter and consists, at least at its tip, of a flexible material which ensures a gentle transport of the IOL. The main body 2 is provided with a guide bush 14 in its end area which forms a stop determining the start position of the piston rod 6 for its axial operation.

Hydraulic stage 4 has a piston annulus 8 and 9 each at both sides of piston 7. The piston annulus 8 is connected to hydraulic stage 5 via a pilot check valve 21 and the piston annulus 9 to the hydraulic chamber 20 via a hole 22. The hydraulic chamber 20 is connected with the piston chamber of piston 17 in the cylinder 18.

The piston annulus 8 is separated from the collecting chamber 15 by a partition wall 16. When connecting the cannula 23 with the main body 2 the film 27 is punctured by the tip of the front tappet section 13 of the tappet 10 establishing the advance connection of the tappet 10 with the IOL 25. Alternatively it is also possible to design the front tappet section 13 of the piston rod 6 as separate component and to deliver it pre-assembled with the cannula 23 and the IOL 25 stored therein. The cannula 23 is provided with a recess in the area of the cannula tip into which an extension of a sealing cap 26 engages to connect the two parts.

FIG. 2 shows the implantation tool with the piston 7 extended by three quarters. This piston position is limited by the guide bush 14 which defines the stroke of the first hydraulic stage 4. To this end, piston rod 6 is moved axially until it is level with the outer surface of the guide bush 14. By the advance movement of the piston 7 the hydraulic fluid is pressed from the first hydraulic stage 4 via the check valve 21 into the hydraulic chamber 20 and the piston annulus 9. The piston annulus 8 is compressed to about ¼ of its volume during this action and piston 17 is extended up to the cap of the cylinder 18. The piston-end tappet section of the tappet 10 at the same time gets into a situation where the partition wall 16 is separated from the end of the center tappet section 12 of tappet 10. The IOL 25 has been transported through the roller path 24 of the cannula 23 up to the point where it enters the capsular bag of the eye.

FIG. 3 shows the IOL 25 leaving the cannula 23 after the advance movement of piston 7 has been completed. The advance movement of piston 7 up to the stop at the partition wall 16 is achieved by operating the second hydraulic stage 5. To this end, the radially movable piston 17 is retracted, the remaining hydraulic fluid of piston annulus 8 is pressed through the open partition wall 16 into the collecting chamber 15 which is equipped with a breather filter and at the same time the piston annulus 9 is supplied with the hydraulic fluid displaced by piston 17.

FIG. 4 shows the cannula 23 detached from the main body 2 of the implantation tool 1. The stored IOL 25 without haptic is shown. The flexible front tappet section 13 of the tappet 10 has punctured the film 27 and is in contact with the IOL 25 ready for the advance movement. The roller path 24 has been filled with lubricant for transport and the advance movement of the IOL 25 can start.

FIG. 5-11 show the individual transport situations of the IOL 25 from the storage phase to the exit from the roller path 24 of the cannula 23.

FIG. 12-14 show how the cannula 23 can be connected with the implantation tool via a T-guide 29 and lateral sliding rails 28 engaging therein.

FIG. 15 shows the implantation tool I with positively connected lubricant fluid container 32. The filling hose 33 is attached to the injector tip of cannula 23 and closed by means of clip 35.

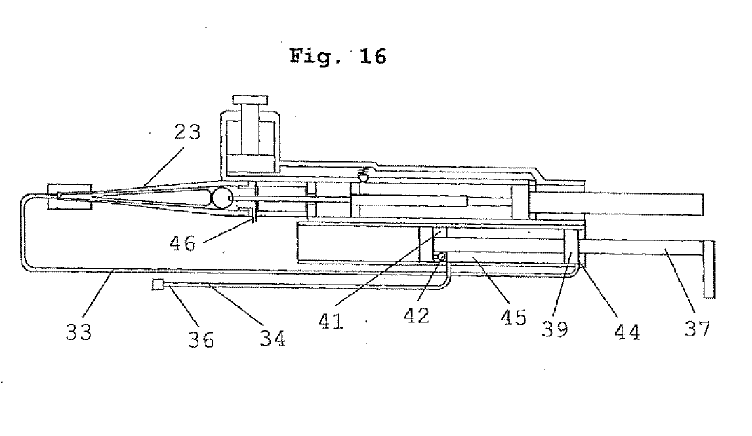

In FIG. 16, the plug 47 has been taken off outlet socket 46, the clip 35 has been removed and the piston rod 37 of the lubricant fluid container 32 extended. This drains the piston annulus 44 via the filling hose 33 into the cannula 23 and at the same time presses the volume of the piston annulus 45 through the hole 42 and via the check valve 43 through the cylinder parting wall 41 into the other piston annulus 45^{λ}. The lubricant fluid 48 transferred this way displaces the sterile storage fluid through the outlet socket 46 of the cannula 23.

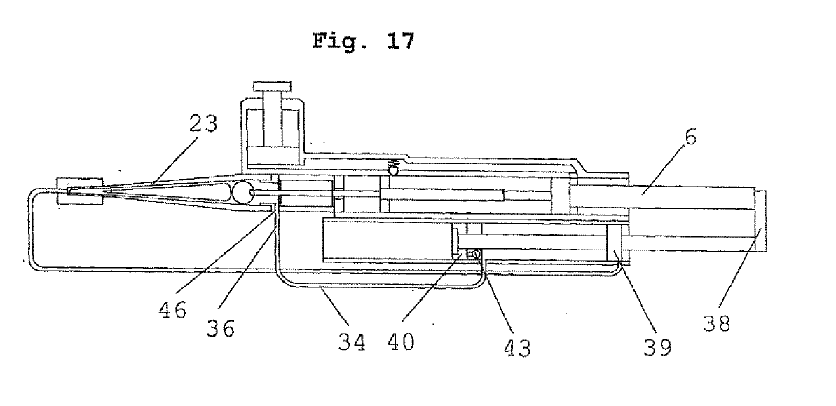

In FIG. 17 it is visible that the angled end 38 of the piston rod 37 is swung upwards and brought into contact with the end of piston rod 6. Next, the filling hose 34 is connected with the outlet socket 46 at the rear end of cannula 23 and the clip 36 is removed.

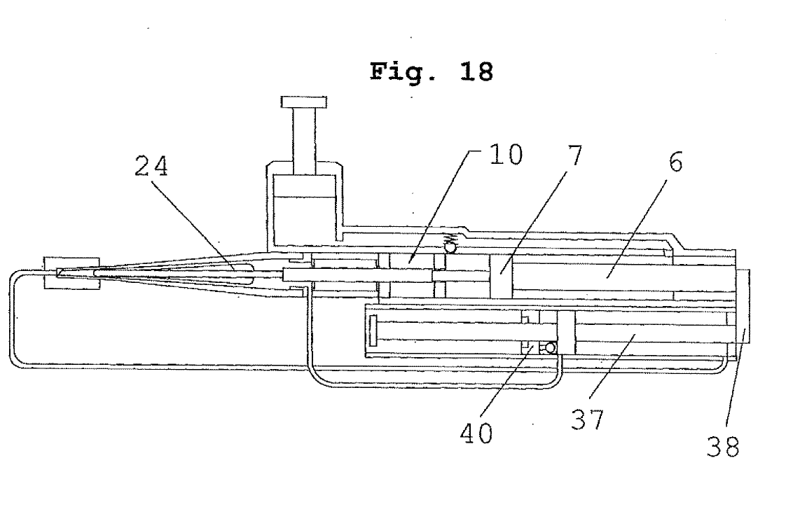

FIG. 18 shows the piston rod 37 retracted again, which has taken piston rod 6 along via its angled end 38 up to the stop at guide bush 14. This causes lubricant fluid 48 to be extracted via the filling hose 33 and at the same time lubricant fluid 48 to be introduced under pressure through the outlet socket 46 behind the IOL 25 via the filling hose 34. This pushing process causes hydrostatic underpressure to build up in front of IOL 25 and hydrostatic overpressure behind IOL 25. IOL 25 is thus transported through the roller path 24 into its rolled-up exit position by applying only little static pressure via piston rod 6 and piston 7 as well as the three-stage tappet 10. To allow this process to be executed smoothly the piston is arranged as loose piston 40 slideably on piston rod 7.

After completion of this cycle the filling hoses 33, 34 are removed, the outlet socket 46 is closed by plug 47 and the lubricant fluid container 32 taken off implantation tool 1. The implantation tool 1 prepared such by the medical staff now again corresponds to FIG. 2 and is passed on to the surgeon for the last operating cycle.

It goes without saying that the invention is not limited to the typical example described in the foregoing, that this example can be subject to alterations to details without leaving the bounds of the invention.

### LIST OF REFERENCE SYMBOLS

1 Implantation tool

2 Main body

3 Two-stage hydraulic mechanism

4 First hydraulic stage

5 Second hydraulic stage

6 Piston rod

7 Piston

8 Piston annulus

9 Piston annulus

10 Three-stage tappet

11 Piston-end tappet section of tappet

12 Center tappet section of tappet

13 Front tappet section of tappet

14 Guide bush

15 Collecting chamber with breather filter

16 Partition wall

17 Piston

18 Cylinder

19 Connection

20 Hydraulic chamber

21 Check valve

22 Hole

23 Cannula

24 Roller path

25 IOL

26 Sealing cap

27 Film

28 Sliding rail

29 T-guide

30 Recess

31 Extension

32 Lubricant fluid container

33 Filling hose

34 Filling hose

35 Clip

36 Clip

37 Piston rod

38 Angled end of piston rod

39 Piston

40 loose piston

41 Cylinder parting wall

42 Hole

43 Check valve

44 Piston annulus

45 Piston annulus

45′ Piston annulus

46 Outlet socket

47 Plug

48 Lubricant fluid

1. Surgical implantation tool for implantation of a deformable intracolar lens made of soft foldable material, particularly silicone or acrylic into an eye, with a tubular main body, preferably with a tubular implant tool which has its internal diameter adapted to the lens, with which the lens can be inserted into the lens capsule of the eye through an incision in the eye and the implantation tool provided with a tappet and with a pushing device for pushing the lens out of the implant tool by means of the tappet wherein the pushing device is provided with a two-stage hydraulic mechanism (3) as drive for the tappet.

2. Implantation tool according to claim 1 wherein the pushing device consists of a rearward piston rod (6), and a piston (7) arranged about in the center, to which the tappet (10) is connected upstream.

3. Implantation tool according to claim 1 wherein the tappet (10) is of three-stage design.

4. Implantation tool according to claim 3 wherein the piston-end tappet section (11) of the tappet (10) has a smaller diameter than the center tappet section (12) of the tappet (10) and the front tappet section (13) has a smaller diameter than the piston-end tappet section (11).

5. Implantation tool according to claim 4 wherein the front tappet section (13) of the tappet (10) is supported separatedly from the tappet and slidably in a cannula (23) preferably forming the implant tool.

6. Implantation tool according to claim 4 wherein the front tappet section (13) of the tappet (10) preferably consists of flexible material.

7. Implantation tool of claim 1 wherein the pushing device is guided in a tubular main body (2) forming the first hydraulic stage (4).

8. Implantation tool of claim 1 wherein the first hydraulic stage (4) in the main body (2) at the rearward end is provided with a relatively long guide bush (14) for the piston rod (6).

9. Implantation tool of claim 1 wherein a collecting chamber (15) with breather filter is connected upstream of the first hydraulic stage (4) in the main body (2).

10. Implantation tool of claim 1 wherein a piston (17) to be operated radially incorporating the second hydraulic stage (5) is mounted radially on the main body (2).

11. Implantation tool of claim 1 wherein the first hydraulic stage (4) is protected from the second hydraulic stage (5) by a check valve (21).

12. Implantation tool of claim 1 wherein the first hydraulic stage (4) is connected with the second hydraulic stage (5) via a hole (22).

13. Implantation tool according to claim 7 wherein the main body (2) is designed such that a cannula (23) is connectable or connected to it by means of a simple connection (19).

14. Implantation tool according to claim 13 wherein the intraocular lens (25) to be implanted is or can be positioned in the cannula (23).

15. Implantation tool according to claim 13 wherein the cannula (23) comes provided with a roller path (24).

16. Implantation tool according to claim 15 wherein the roller path (24) tapers like a nozzle.

17. Implantation tool according to claim 13 wherein a detachable or removable sealing cap (26) is provided before the exit area of the roller path (24).

18. Implantation tool according to claim 13 wherein the rearward area of the cannula (23) is closed by a film (27) which can be punctured easily.

19. Implantation tool according to claim 13 wherein the interior of the cannula (23) is preferably filled with distilled water.

20. Implantation tool according to claim 17 wherein a lubricant fluid can be introduced through the central opening of the cannula (23).

21. Implantation tool according to claim 1 wherein the main body (2) is made from medical plastics, stainless steel or preferably titanium or a titanium alloy sterilizable in an autoclave.

22. Implantation tool according to claim 21 wherein the main body (2) is reusable after sterilization.

23. Implantation tool according to claim 2 wherein a partial area of the first hydraulic stage (4) is limited by retracting the rearward piston rod (7) up to the end-side stop and the second hydraulic stage (5) is filled with the hydraulic fluid of said partial area of the first hydraulic stage (4).

24. Implantation tool according to claim 23 wherein the connection to the collecting chamber (15) is open in the transition area between the first and second hydraulic stages.

25. Implantation tool of claim 22 wherein the second hydraulic stage (5) is being loaded by the piston (17) to be operated radially, the piston annulus (9) is filled from the second hydraulic stage (5) and the piston 87) is extended until it contacts the foremost end stop.

26. Implantation tool according to claim 25 wherein the radially acting piston (17) is extended, then the pilot check valve (21) is opened, the piston (17) retracted again thus returning the two-stage hydraulic mechanism (3) to its original position.

27. Implantation tool according to claim 1 wherein a lubricant fluid container (32) is detachably connected with the implantation tool (1).

28. Implantation tool with lubricant fluid container according to claim 27 wherein the lubricant fluid container (32) is connected to the cannula (23) via two filling hoses (33, 34).

29. Implantation tool with lubricant fluid container according to claim 28 wherein the filling hose (33) is connected to the tip of the cannula (23) and the filling hose (34) is connected via an outlet socket (46). to the end of the cannula.

30. Lubricant fluid container for an implantation tool according to claim 27 wherein the lubricant fluid container (32) consists of a cylinder being open at one end.

31. Lubricant fluid container according to claim 30 wherein the piston rod (37) is bipartite and has a piston (39) in the center and a loose piston (40) at one end.

32. Lubricant fluid container according to claim 31 wherein the extendable piston rod end is provided with an end angled by 90°.
